(19) United States
(12) Patent Application Publication
(10) Pub. No.: US 2026/0076786 A1
(43) Pub. Date: Mar. 19, 2026

(54) COLOR-CHANGING DENTURE MOLD

(71) Applicant: Crown Warranty, LLC, Mayfield Heights, OH (US)

(72) Inventors: Adam Jaffe, Aurora, OH (US); Sam Jaffe, Pepper Pike, OH (US)

(21) Appl. No.: 19/332,843

(22) Filed: Sep. 18, 2025

Related U.S. Application Data
(60) Provisional application No. 63/696,362, filed on Sep. 18, 2024.

(51) Int. Cl. A61C 13/01; A61K 6/887

(52) U.S. Cl. CPC A61C 13/01; A61K 6/887; A61C 2201/002

(57) ABSTRACT

A denture set, comprising an upper denture mold and a lower denture mold, wherein the upper denture mold and lower denture mold comprise a heat moldable material that is configured to be malleable when heated, and wherein the upper denture mold and lower denture mold comprise a thermochromic material configured to change colors when heated.

### RELATED APPLICATION

This application claims priority to U.S. Provisional Patent Application No. 63/696,362, filed on September 18, 2024, and entitled “COLOR-CHANGING DENTURE MOLD”, the entirety of which is incorporated herein by reference.

### BACKGROUND

Dentures are removable replacements for missing teeth and surrounding tissues. Dentures comprise artificial teeth attached to a mold that snugly fit into a patent’s mouth. Typically, dentures are custom-made to fit an individual’s mouth and can help improve chewing, speaking, and appearance.

The manufacturing process for dentures can be expensive and time consuming. Creating dentures is a detailed process that can take several weeks. This includes multiple appointments for molds, fittings, and adjustments, which can be inconvenient for patients. Additionally, achieving a precise fit can be difficult. Small inaccuracies in the molds can lead to discomfort or poor function, necessitating adjustments or remakes.

### SUMMARY

The following is a brief summary of subject matter that is described in greater detail herein. This summary is not intended to be limiting as to the scope of the claims.

Various systems and methods described herein relate to a dental set comprising an upper denture mold and a lower denture mold. The upper denture mold and lower denture mold comprise an upper heat moldable portion and a lower heat moldable portion, respectively. The upper heat moldable portion and the lower heat moldable portion are formed of a heat moldable material configured to be malleable after exposure to heat. The upper heat moldable portion and lower heat moldable portion also comprise a thermochromic material configured to change colors after exposure to heat, indicating that the upper heat moldable portion and lower heat moldable portion are malleable and ready to be formed.

After the upper denture mold and lower denture mold are exposed to heat, the upper denture mold and lower denture mold are inserted into a mouth of a user, where the upper denture mold and lower denture mold may be pushed, molded, or moved to contour to the shape of the mouth of the user. After conforming to the mouth, the upper denture mold and lower denture mold form ridges, curves, bumps, and other features that allow the upper denture mold and lower denture mold to fit snugly in the mouth.

The upper denture mold and the lower denture mold also comprise upper teeth and lower teeth, respectively. The upper teeth and lower teeth are integrally formed with the upper heat moldable portion and the lower heat moldable portion, respectively..

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

FIGS. 1-6 are perspective views of a denture set comprising an upper denture mold and a lower denture mold with a thermochromic material.

FIG. 7 is a methodology of preparing a denture set.

### DETAILED DESCRIPTION

Various systems pertaining to a denture set are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

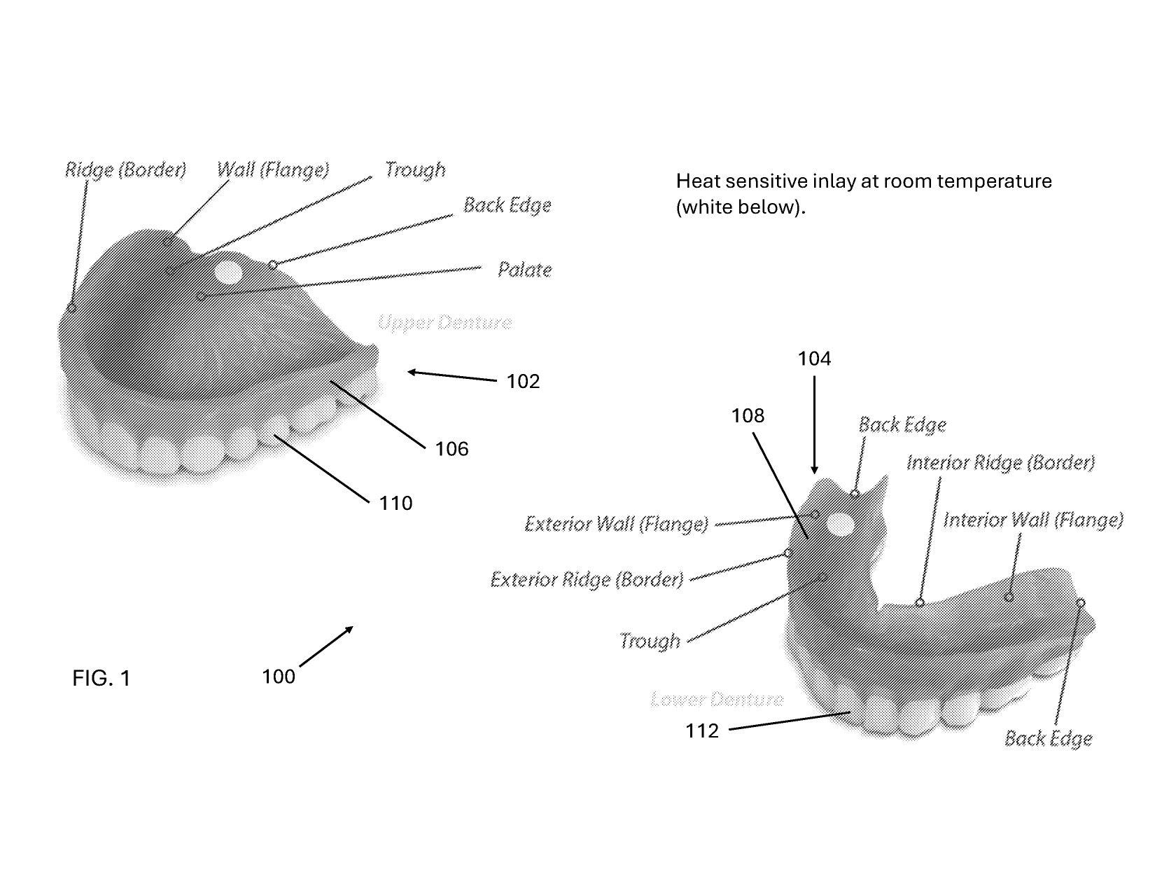

With reference to FIG. 1, a denture set 100 comprising an upper denture mold 102 and a lower denture mold 104 is illustrated. The upper denture mold 102 and the lower denture mold 104 comprise an upper heat moldable portion 106 and a lower heat moldable portion 108, respectively. The upper heat moldable portion 106 and lower heat moldable portion 108 are formed of a heat moldable material that is configured to be malleable after exposure to heat, such as in hot water or in a temperature-controlled oven. In an embodiment, the heat moldable material is polypropylene. In another embodiment, the heat moldable material is any suitable thermoplastic material, such as ultra-high molecular weight polyethylene. After the upper heat moldable portion 106 and lower heat moldable portion 108are heated and removed the heat, the upper heat moldable portion 106 and lower heat moldable portion 108 cure after a given time (e.g., 5 to 10 minutes) depending on the type of heat moldable material, the temperature of the surroundings of the upper heat moldable portion 106 and lower heat moldable portion 108once the upper heat moldable portion 106 and lower heat moldable portion 108 are removed from the heat, and other factors. When desired, the upper heat moldable portion 106 and lower heat moldable portion 108may be re-heated to be malleable and cured again into a different shape or configuration.

The upper heat moldable portion 106 and lower heat moldable portion 108also comprise a thermochromic material configured to change colors when heated, indicating that the upper heat moldable portion 106 and lower heat moldable portion 108are malleable and ready to be formed. The thermochromic material can be a color-changing material, such as heat-sensitive colorant. For instance, the thermochromic material may be mixed with polypropylene. The thermochromic material can exhibit differing colors depending upon the temperate of the thermochromic material. Accordingly, the upper heat moldable portion 106 and lower heat moldable portion 108 can turn into a plurality of different colors depending on the characteristics of the thermochromic material included as part of the upper heat moldable portion 106 and lower heat moldable portion 108. In an example, the thermochromic material of the upper heat moldable portion 106 and lower heat moldable portion 108 is pink before being exposed to heat (e.g., hot water, temperature-controlled oven) and the thermochromic material of the upper heat moldable portion 106 and lower heat moldable portion 108 becomes yellow after being exposed to heat (e.g., when a temperature of the thermochromic material reaches a threshold temperature); following this example, the thermochromic material returns to be pink after curing (e.g., after removal from the heat and lowering of the temperature). In another example, the thermochromic material of the upper heat moldable portion 106 and lower heat moldable portion 108 is gum-colored (i.e., coral) before being exposed to heat and the thermochromic material of the upper heat moldable portion 106 and lower heat moldable portion 108 becomes blue after being exposed to heat (e.g., when a temperature of the thermochromic material reaches a threshold temperature); pursuant to this example, the thermochromic material returns to be gum-colored after curing (e.g., after removal from the heat and lowering of the temperature).

Once the upper heat moldable portion 106 and lower heat moldable portion 108reach a specified temperature (e.g., between 140-240 degrees Fahrenheit, the threshold temperature), the thermochromic material of the upper heat moldable portion 106 and lower heat moldable portion 108 changes colors (e.g., from pink to yellow). When the thermochromic material of the upper heat moldable portion 106 and lower heat moldable portion 108 changes colors, the upper heat moldable portion 106 and lower heat moldable portion 108 are malleable and ready to be formed, and are removed from the heat. After the upper heat moldable portion 106 and lower heat moldable portion 108cool off and cure, the color of the thermochromic material of the upper heat moldable portion 106 and lower heat moldable portion 108can revert to the upper heat moldable portion’s 106 and lower heat moldable portion’s 108 original color. For example, if the thermochromic material of the upper heat moldable portion 106 is yellow after being exposed to heat, the thermochromic material of the upper heat moldable portion 106 reverts to pink after curing. Conversely, after the upper heat moldable portion 106 and lower heat moldable portion 108cool off and cure, the color of the thermochromic material of the upper heat moldable portion 106 and lower heat moldable portion 108 may not revert to the original color. For example, if the thermochromic material of the upper heat moldable portion 106 is yellow after being exposed to heat, the thermochromic material of the upper heat moldable portion 106 may stay yellow after curing in order to indicate that the upper heat moldable portion 106 was exposed to heat or a specific temperature (e.g., 160 degrees Fahrenheit, etc.).

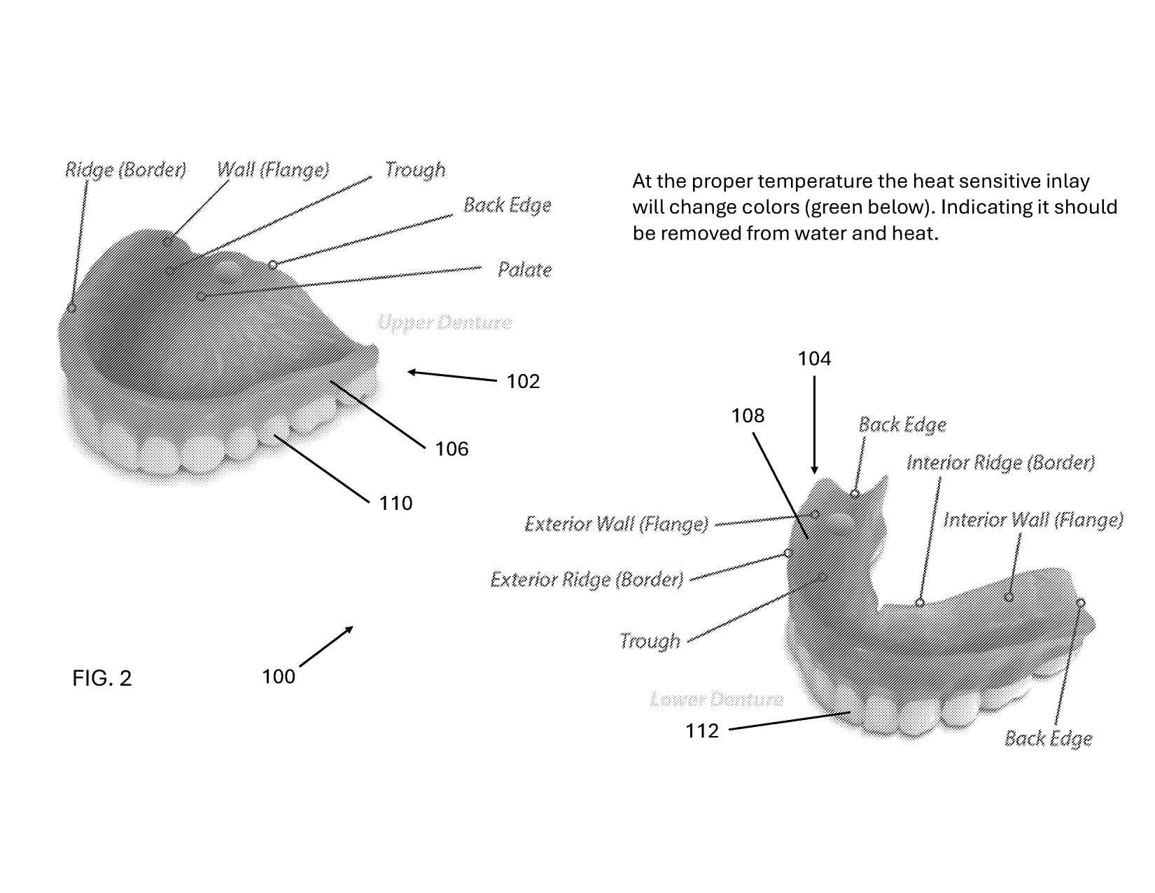

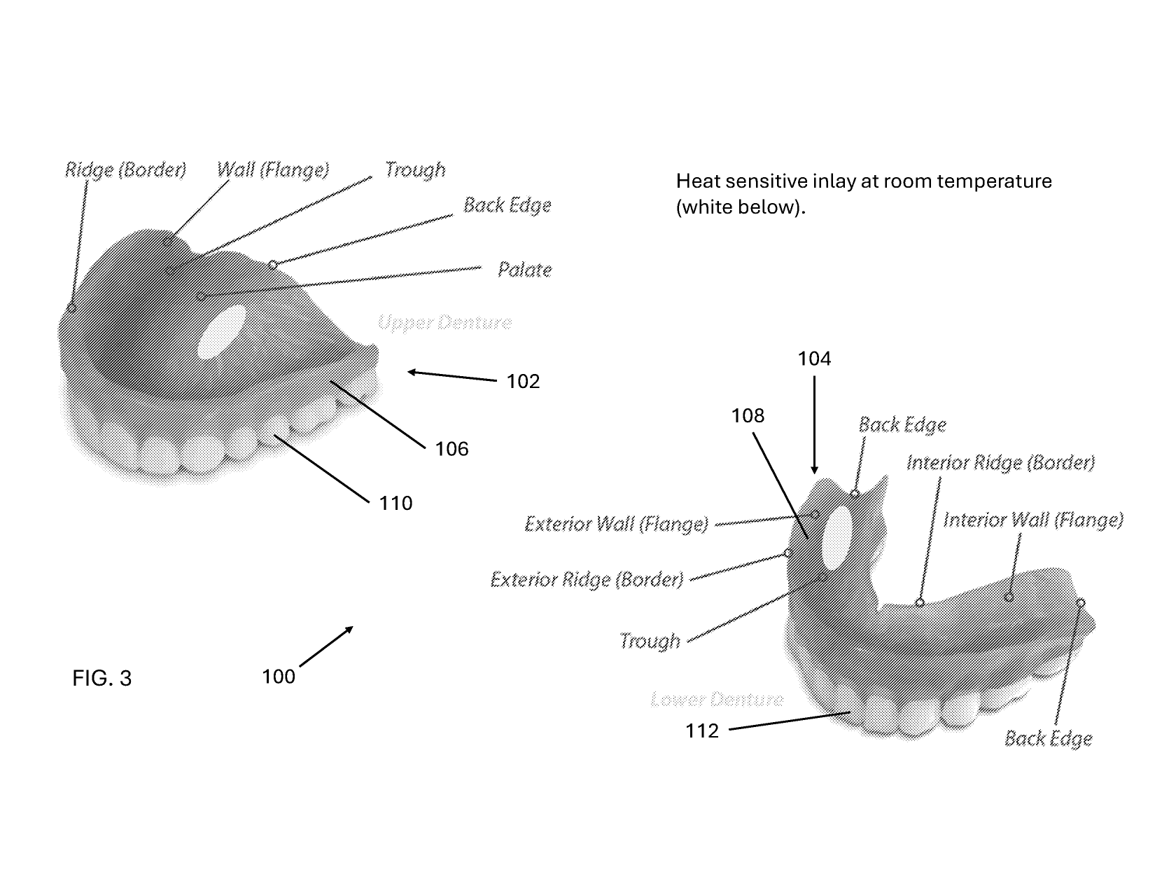

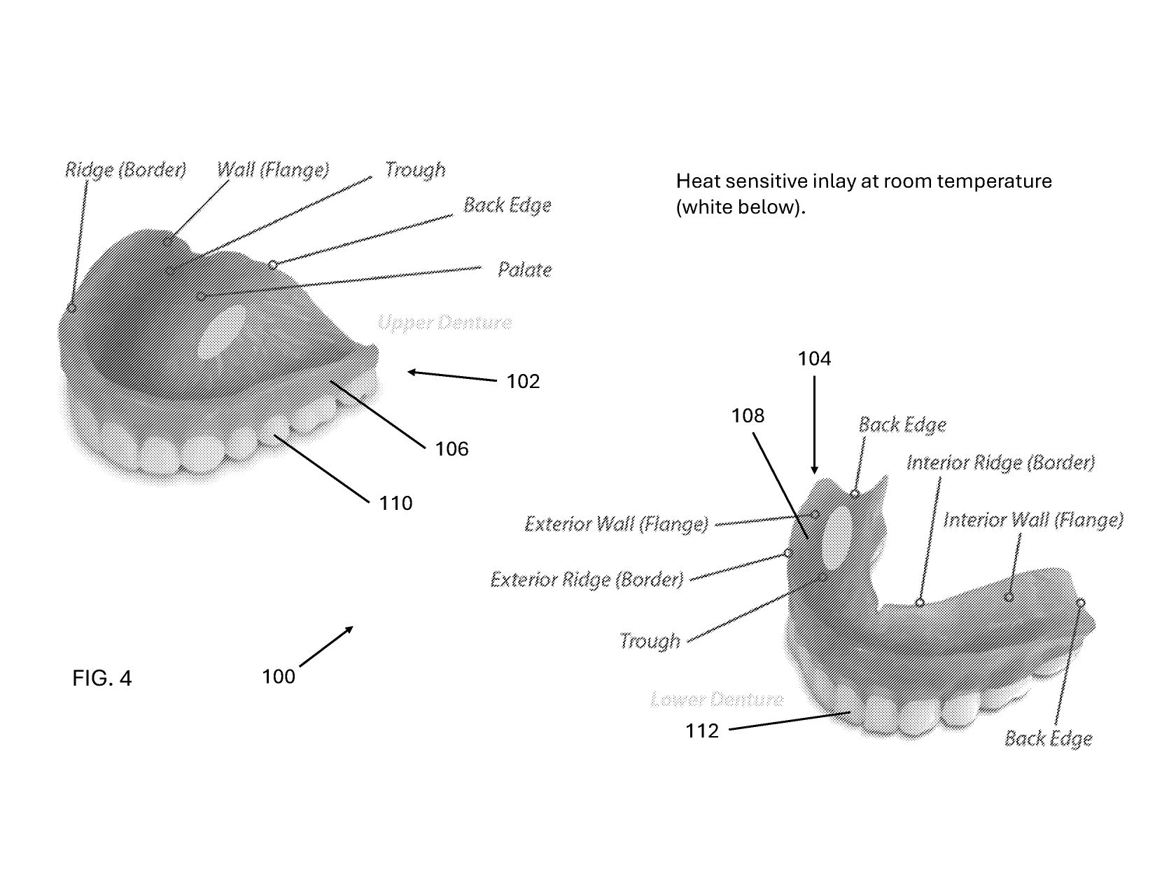

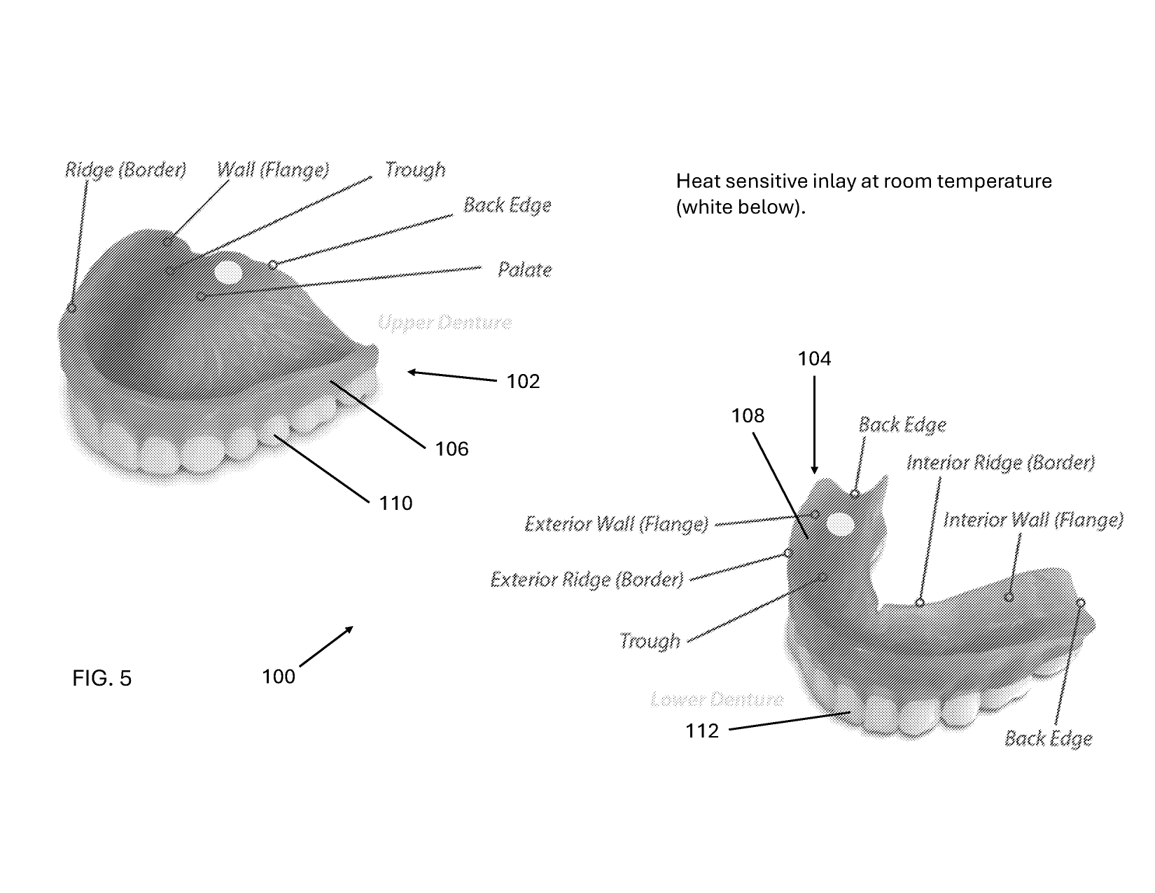

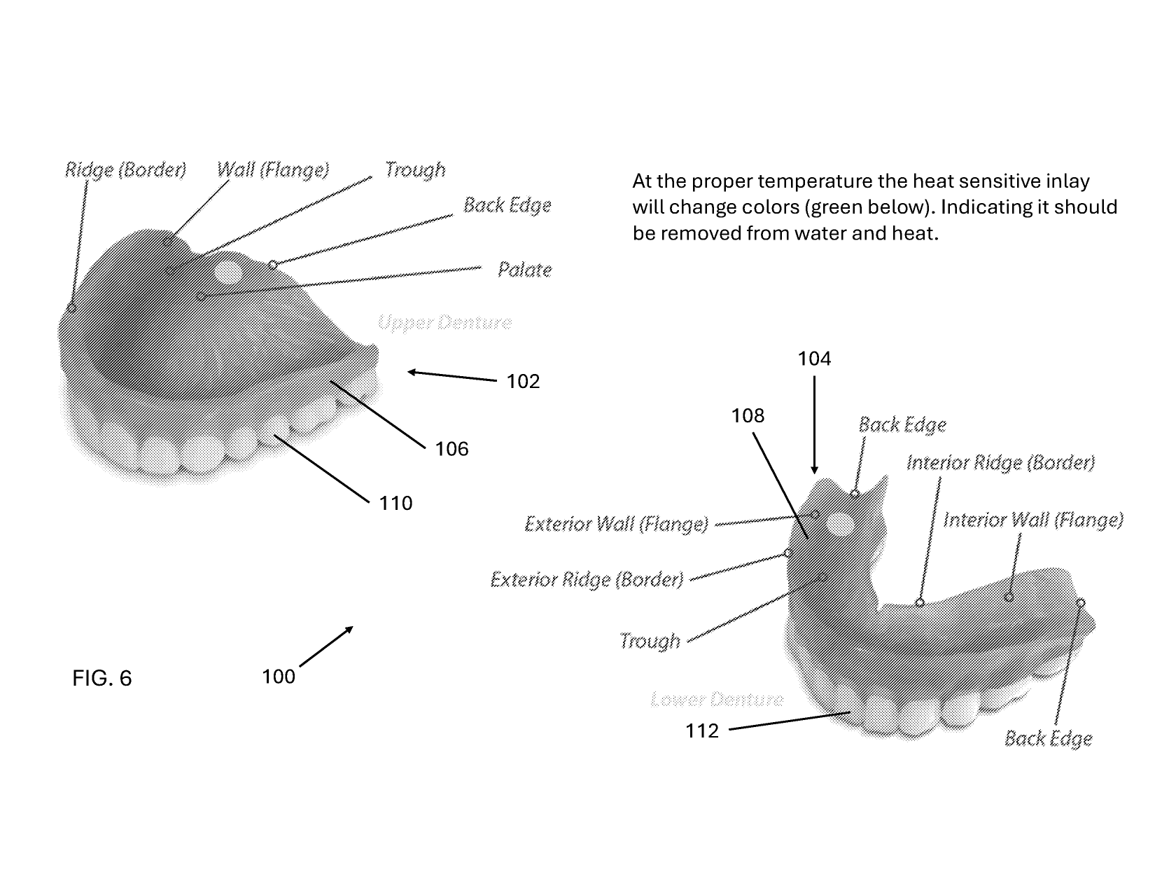

The thermochromic material can be located throughout the upper heat moldable portion 106 and lower heat moldable portion 108. Alternatively, in some examples, the thermochromic material is located on a palate of the upper heat moldable portion 106 and a flange of the lower heat moldable portion 108. In other examples, the thermochromic material is located on a trough of the upper heat moldable portion 106 and a trough of the lower heat moldable portion 108. FIGS. 1-6 comprise circles and ovals with different colors on the palate and trough of the upper heat moldable portion 106 and the flange and trough of the lower heat moldable portion 108, approximating the locations of where the thermochromic material can be located on the upper heat moldable portion 106 and lower heat moldable portion 108. It is submitted that while the circles and ovals are indicative of the locations of the thermochromic material on the upper heat moldable portion 106 and lower heat moldable portion 108, the thermochromic material may be organized in a plurality of ways in those locations (e.g., in a square, in a rectangle, across the entirety of the trough/flange/palate, as dots, etc.). It is also submitted that the circles and ovals are different colors (e.g., white, green, yellow, and pink) indicating the plurality of different colors that the thermochromic material can change to when exposed to heat. For example, the circles of FIGS. 1, 2, 5, and 6 are white, green, yellow, and pink, respectively, and the ovals of FIGS. 3 and 4 are yellow and pink, respectively.

The upper heat moldable portion 106 comprises several features, such as a ridge, a wall, a trough, a back edge, and a palate, which are moldable after the upper heat moldable portion 106 is exposed to heat. Accordingly, when the upper heat moldable portion 106 is exposed to heat, the ridge, wall, trough, back edge, and palate are moldable to expand, contract, flex, or otherwise adapt to surface area properties of a top area of a mouth of a user. After the upper heat moldable portion 106 cools and cures, the ridge, wall, trough, back edge, and palate retain their shapes to fit snugly against the top area of the mouth.

The lower heat moldable portion 108 comprises several features, such as an exterior wall, an exterior ridge, a trough, a first back edge, a second back edge, an interior ridge, and an interior wall, which are moldable after the lower heat moldable portion 108 is exposed to heat. Accordingly, when the lower heat moldable portion 108 is exposed to heat, the exterior wall, exterior ridge, trough, first back edge, second back edge, interior ridge, and interior wall are moldable to expand, contract, flex, or otherwise adapt to surface area properties of a bottom area of a mouth of a user. After the lower heat moldable portion 108 cools and cures, the exterior wall, exterior ridge, trough, first back edge, second back edge, interior ridge, and interior wall retain their shapes to fit snugly against the top area of the mouth.

The upper denture mold 102 and the lower denture mold 104 also comprise upper teeth 110 and lower teeth 112, respectively. The upper teeth 110 and lower teeth 112 are integrally formed with the upper heat moldable portion 106 and the lower heat moldable portion 108, respectively. The upper teeth 110 and lower teeth 112 may be formed of any suitable rigid material, including porcelain, composite resin, ceramic, acrylic, etc. The upper teeth 110 also comprise an upper base and the lower teeth 112 also comprise a lower base. The upper base and the lower base may be formed of a suitable rigid material, including porcelain, composite resin, ceramic, acrylic, etc., and/or formed of a flexible denture base. In an example, the upper teeth 110, lower teeth 112, upper base, and lower base comprise the same material, such as acrylic. The upper heat moldable portion 106 can be secured to the upper teeth 110 in a plurality of methods, including using adhesives, heating, machining, injecting, etc. Additionally, the lower heat moldable portion 108 can be secured to the lower teeth 112 in a plurality of methods, including using adhesives, heating, machining, injecting, etc. After the upper heat moldable portion 106 has been attached to the upper teeth 110 and the lower heat moldable portion 108 has been attached to the lower teeth 112, the upper teeth 110 and lower teeth 112 are polished.

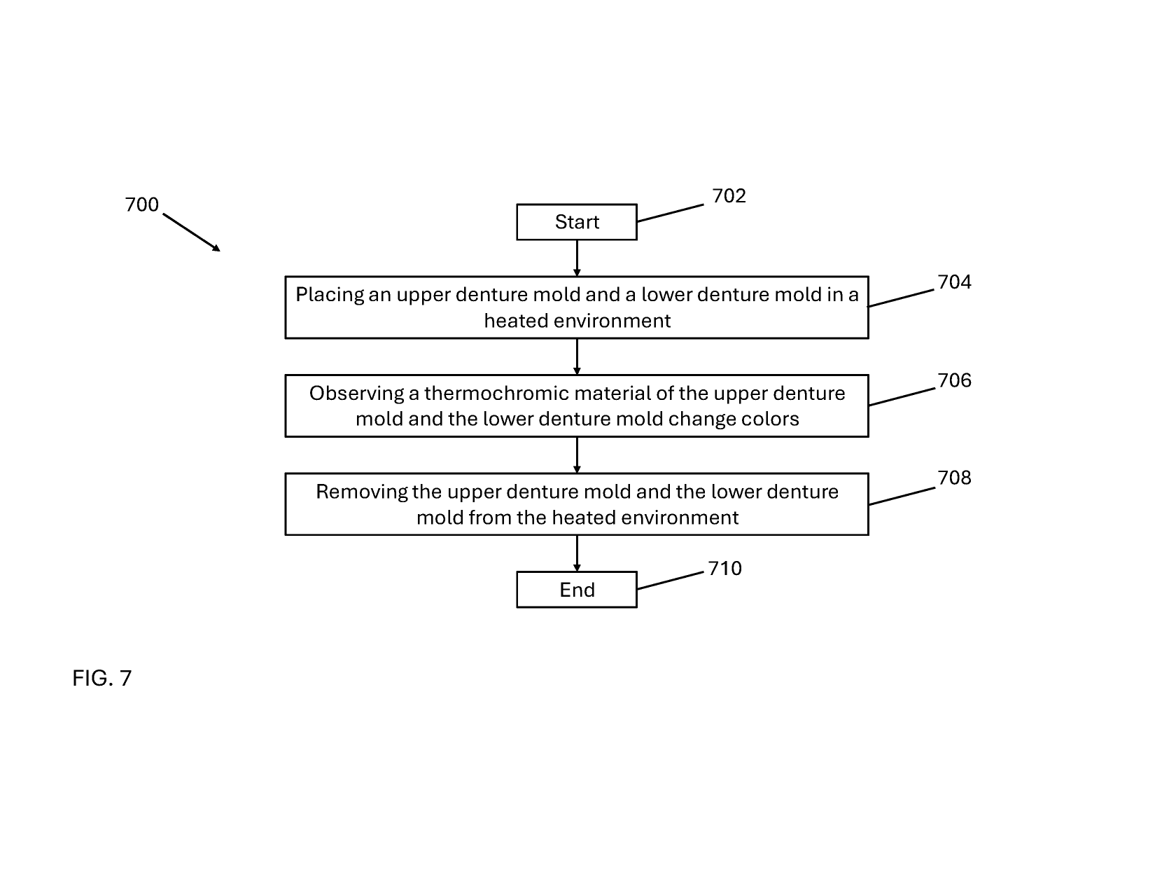

FIG. 7 illustrates an exemplary methodology relating to the preparation of color-changing denture molds. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement a methodology described herein.

Now referencing FIG. 7, a method of preparing color-changing denture molds 700 is shown. The method starts at 702, and at 704, an upper denture mold and a lower denture mold are placed in a heated environment. At 706, a thermochromic material of the upper denture mold and the lower denture mold is observed changing colors. At 708, the upper denture mold and the lower denture mold are removed from the heated environment, and at 710, the method ends. It is to be appreciate that the heated environment could be any suitable heated environment, including hot water or a temperature-controlled oven.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A denture set, comprising:
an upper denture mold and a lower denture mold,
wherein the upper denture mold comprises an upper heat moldable portion formed of a heat moldable material that is configured to be malleable when heated,
wherein the lower denture mold comprises a lower heat moldable portion formed of the heat moldable material that is configured to malleable when heated, and
wherein the upper heat moldable portion and lower heat moldable portion comprise a thermochromic material configured to change colors based on a temperature of the thermochromic material.

2. The denture set of claim 1, further comprising:
upper teeth and lower teeth,
wherein the upper teeth are attached to the upper heat moldable portion and the lower teeth are attached to the lower heat moldable portion.

3. The denture set of claim 1, wherein the heat moldable material is polypropylene.

4. The denture set of claim 1, wherein the thermochromic material is a heat-sensitive colorant.

5. The denture set of claim 1, wherein the thermochromic material is located on a palate of the upper heat moldable portion and a flange of the lower heat moldable portion.

6. The denture set of claim 1, wherein the thermochromic material is located on a palate of the upper heat moldable portion and a trough of the lower heat moldable portion.

7. The denture set of claim 1, wherein the thermochromic material is located on a trough of the upper heat moldable portion and a trough of the lower heat moldable portion.

8. The denture set of claim 1, wherein the thermochromic material is located on a trough of the upper heat moldable portion and a flange of the lower heat moldable portion.

9. The denture set of claim 1, wherein the thermochromic material of the upper heat moldable portion and the lower heat moldable portion is yellow before being exposed to heat.

10. The denture set of claim 1, wherein the thermochromic material of the upper heat moldable portion and the lower heat moldable portion is pink after being exposed to heat.

11. The denture set of claim 1, wherein the thermochromic material of the upper heat moldable portion and the lower heat moldable portion is a first color before being exposed to heat.

12. The denture set of claim 11, wherein the thermochromic material of the upper heat moldable portion and the lower heat moldable portion is a second color after being exposed to heat.

13. The denture set of claim 12, wherein the thermochromic material of the upper heat moldable portion and the lower heat moldable portion reverts from the second color to the first color after cooling.

14. The denture set of claim 12, wherein the thermochromic material of the upper heat moldable portion and lower heat moldable portion remains the second color after cooling.

15. The denture set of claim 1, wherein the upper heat moldable portion and lower heat moldable portion are configured to be re-heated.

16. The denture set of claim 1, wherein the thermochromic material of the upper heat moldable portion and the lower heat moldable portion changes color when heated to a temperature of 160 degrees Fahrenheit.

17. The denture set of claim 1, wherein the upper denture mold and lower denture mold are configured to be heated in a temperature-controlled oven.

18. A method of preparing a denture set, comprising:
placing an upper denture mold and a lower denture mold in a heated environment;
observing a thermochromic material of the upper denture mold and the lower denture mold change colors; and
removing the upper denture mold and the lower denture mold from the heated environment.

19. The method of claim 18, wherein the heated environment is hot water.
